(19) United States
(12) Patent Application Publication
(10) Pub. No.: US 2025/0360221 A1
(43) Pub. Date: Nov. 27, 2025

(54) ESTERS OF BETA ENDORPHIN

(71) Applicant: Joel Steven Goldberg, Hillsborough, NC (US)

(72) Inventor: Joel Steven Goldberg, Hillsborough, NC (US)

(21) Appl. No.: 19/241,513

(22) Filed: Jun. 18, 2025

(51) Int. Cl. A61K 47/64

(52) U.S. Cl. CPC A61K 47/64

(57) ABSTRACT

Chronic intractable pain is a global health issue, highlighting the need for a potent μ-agonist analgesic with minimal major side effects. Intrathecal administration of β-endorphin in humans produces significant analgesia without respiratory depression or tolerance. Earlier animal studies concluding that chronic administration of β-endorphin results in tolerance and respiratory depression may not be applicable to humans due to differences between human and animal μ-receptors and β-endorphins. It is anticipated that β-endorphin functions as a GPCR biased ligand, similar to ligand binding in endocrinopathies that do not usually produce tolerance. Ester prodrugs of β-endorphin are predicted to cross the human blood-brain barrier (BBB), undergo hydrolysis to β-endorphin and a non-toxic metabolite, and provide substantial analgesia with low toxicity and without major side effects. The synthesis of lead prodrugs is described. Compared to current pharmaceutical development expenditures, the risks associated with this research are considered low.

### CROSS-REFERENCE TO RELATED APPLICATIONS

This application claims the benefit of U.S. application Ser. No. 17/526,163, Ser. No. 15/438,955, and Ser. No. 13/613,736 which are incorporated by reference. Incorporation by reference is the Sequence Listing XML file named amino acids.xml which was created on Aug. 20, 2025, is 8,225 bytes, and is supported by the amino acid sequences in the description of drawings. SEQ ID NO: 1 is Mus musculus mu-type opioid receptor(OPMR_MOUSE), SEQ ID NO: 2 is Rattus norvegicus mu-type opioid receptor(OPRM_Rat), SEQ ID NO: 3 is Homo sapiens mu-type opioid receptor(OPRM_Human), SEQ ID NO: 4 is Homo sapiens beta endorphin(COLI_HUMAN), SEQ ID NO: 5 is Rattus norvegicus beta endorphin(COLI_RAT), SEQ ID NO: 6 is Mus musculus beta endorphin(COLI_MOUSE), and SEQ ID NO: 7 is Camelus dromedarius beta endorphin(COLI-CAMDR). No new matter has been added to the specification.

### FEDERALLY SPONSORED RESEARCH AND DEVELOPMENT

Not applicable

### BACKGROUND OF THE INVENTION

Chronic pain, encompassing inflammatory, neuropathic, and cancer-related pain, significantly impacts the aging global population. There is an unmet need for a potent μ-opioid receptor agonist with minimal major adverse side effects including respiratory depression, tolerance, physical dependence, and addiction. There are other side effects from opioid administration such as constipation, urinary retention, pruritus, immunosuppression, hypogonadism, hyperalgesia, sedation, and mild cognitive dysfunction but these are of secondary concern compared to the major previously cited side effects.

Despite substantial investment in the development of such a μ-agonist, success has been limited. Recent computer-designed μ-agonists like PZM21 and TRV130 have not met these needs. [1, 2] A G-protein-coupled receptor (GPCR) biased μ-agonist with minimal β-arrestin translocation, intracellular phosphorylation, and internalization remains undeveloped. Notably, the endogenous opioid peptide β-endorphin, which is 20 times more potent than morphine, may likely meet these criteria. Currently, natural design appears to surpass computer-aided drug design.

β-endorphin is primarily synthesized in the central nervous system (CNS) through the cleavage of pro-opiomelanocortin (POMC). α and γ endorphins are not analgesic. β-endorphin (1-27) is less potent than β-endorphin (1-31) suggesting that the complete endogenous peptide will achieve the best fit to the GPCR. Both the N- and C-terminal sequences are essential for its potent analgesic effects, and β-endorphin is quite stable and resistant to many peptidases.[3] Intrathecal administration of 3 mg of β-endorphin to patients with severe cancer pain resulted in significant analgesia lasting approximately 33.4 hours without major adverse effects.[4] Epidural injections also provided analgesia for a shorter duration of 19.5 hours, again without major side effects. [5] In two patients suffering from cancer pain continuous intrathecal infusion of β-endorphin for two weeks provided profound analgesia, and there was no evidence of tolerance or respiratory depression.[6] These case studies in humans support the underlying hypothesis of this invention.

Clinical research confirms that tolerance from sustained endogenous GPCR agonists does not usually occur with hypersecretory conditions such as pheochromocytoma (norepinephrine and epinephrine), acromegaly (somatotropin), pituitary hyperthyroidism (TSH), hyperparathyroidism (PTH), Cushing's disease (ACTH), and carcinoid syndrome (serotonin). Furthermore, stable dose endocrine supplementation for deficiency states with endogenous ligands or mimetics that bind to GPCR is a mainstay of treatment sometimes for a lifetime without evidence of tolerance. This implies that these endogenous agonists are GPCR biased with minimal β-arrestin translocation, intracellular phosphorylation, or internalization which is relevant to β-endorphin. It is hypothesized that β-endorphin administration does not produce desensitization or tolerance which is common with other μ-receptor agonists and does not produce respiratory depression. In humans there is an imperative to prove or disprove this hypothesis.

### μ1, μ1, μ3 Opioid Receptors

Although μ1, μ2, μ3 opioid receptors are GPCR there are distinctions that are relevant to this invention. In animals, there are multiple spice variants of the μ1 receptor. [7] Ligand binding to μ1 receptors with opioid peptides and opioids alkaloids produce profound analgesia. Ligand binding to μ2 receptors produces a different analgesic response with a TRMU-5 agonist.[8] Ligand binding to μ3 receptors with opioid peptides does not produce analgesia but binding with opioid alkaloids does produce analgesia which implies that there is discrimination in mode of action between opioid peptides and opioid alkaloids which may extend not only to analgesia but to adverse effects.[9] Genetic comparisons confirm that the μ-opioid receptor in animals is not identical to the u opioid receptor in humans, and human β-endorphin is not identical to animal β-endorphin. (FIGS. 1-2) It is well know that a single nucleotide substitution can have major effects on phenotype such as in sickle cell disease, phenylketonuria, Marfan's syndrome, β thalassemia, etc. In humans X-ray crystallography docking studies demonstrate that morphine docking sites are not congruent with the β-endorphin docking site. (FIGS. 3-4)

### Peptides Known to Cross the BBB

Contrary to popular belief, peptides can cross the blood brain barrier (BBB) despite not meeting Lipinski's criteria.[10] In humans β-endorphin does not cross the BBB when administered intravenously. Table 1 lists peptides that cross the BBB and their associated Lipinski criteria and comparison to β-endorphin.

TABLE 1
Lipinski Criteria of Peptides Known to
Cross the BBB Compared to β-endorphin

| Peptide | MW | #AA | XLogP3 | HBD | HBA |
|---|---|---|---|---|---|
| Insulin | 5794 | 51 | −12.8 | 78 | 89 |
| Ghrelin | 3370 | 28 | −18.3 | 48 | 51 |
| Leptin | 2004 | 146 | 4.4 | 30 | 50 |
| Amylin | 3903 | 37 | −18.7 | 61 | 61 |
| Orexin-A | 3561 | 33 | −10.1 | 49 | 53 |
| β-endorphin | 3465 | 31 | −13.9 | 48 | 53 |

This invention demonstrates that modification of β-endorphin to increase lipophilicity through esterification and/or through ester attachment of a known transporter could enhance its ability to cross the human BBB as a prodrug. Upon crossing the barrier, it undergoes ester hydrolysis, converting into an active μ-agonist with minimal major adverse effects and a metabolite of low-toxicity in the CNS. The prodrug also referred to as a conjugate can be synthesized with β-endorphin and molecules or their immediate metabolites known to be relatively non-toxic to the CNS include but not limited to ethanol, cholesterol, glucose, docosahexaenoic acid (DHA), linolenic acid or ascorbic acid.

### Oral Absorption of Peptide Drugs

Few peptide drugs are orally absorbed, and developing oral insulin has been an ongoing challenge. Obstacles include instability from stomach acid and enzymes, limited permeability, and varying individual physiology. Attempts to enhance absorption, like enteric coating, enzyme inhibitors, encapsulation, and cyclization, have seen limited success. The main issue remains poor intestinal permeability. Table 2 lists commercially available oral peptide drugs with Lipinski criteria. Developing esters of β endorphin that are orally absorbed would need to be preceded by the development of a successful parenterally administered drug.

TABLE 2
Lipinski Criteria of Orally Absorbed Modified Peptide Drugs

| Peptide | MW | #AA | XLogP3 | HBD | HBA |
|---|---|---|---|---|---|
| Cyclosporin | 1202 | 11 | 7.5 | 5 | 12 |
| A | | | | | |
| Semaglutide | 4114 | 31 | −5.8 | 57 | 63 |
| Octreotide | 1019 | 8 | 1 | 13 | 14 |

### Plasma Degradation

When compared to other opioid peptides β-endorphin is resistant to degradation by proteolytic enzymes.[3] The ester prodrug of β-endorphin will likely be hydrolyzed by esterases in plasma, but this does not limit its use as a CNS analgesic. Many ester drugs, such as procaine, 2-chloroprocaine, heroin, cocaine, sobetirome, etc. have CNS activity and are initially hydrolyzed by esterases in the plasma probably because the rate of hydrolysis is rather slow. (See prior art)

### Ion Trapping Effects

The average pH of CSF is 7.33 while the average pH of plasma is 7.41. The β-endorphin ester prodrug after hydrolysis will preferentially concentrate in the CSF from the generated carboxylic acid ion and from amines.

### CNS Toxicity

The CNS toxicity of β-endorphin esters is predicted to be low. The degradation of the prodrug will produce β-endorphin and a substance known to be minimally toxic to the CNS such as ethanol, cholesterol, glucose, DHA, linolenic acid, or ascorbic acid. It is anticipated that the alcohols of DHA and linolenic acid will be converted in the CNS to the respective carboxylic acids by dehydrogenases.

### Respiratory Depression

There is no evidence of respiratory depression from intrathecal or epidural administration of β-endorphin to humans.[4-6] Contrarily there are in vivo reports of respiratory depression in animals with intracerebroventricular administration.[11-13] Genetic comparisons confirm that the μ-opioid receptor in animals is not identical to the μ-opioid receptor in humans and human β-endorphin is not identical to animal β-endorphin. (FIGS. 1-2)

### Tolerance

There are no known studies of β-endorphin tolerance in humans, but continuous intrathecal administration for two weeks in two patients did not demonstrate tolerance.[6] In animals, tolerance develops after intracerebroventricular injection of β-endorphin and after guinea pig ileum assays treated with β-endorphin.[14-16] As previously stated there is very compelling evidence that animal studies conducted decades ago may not translate to pharmacodynamics in humans.

### Major Considerations in Design of Esters of β-Endorphin

The major factors in the design of ester prodrugs of β-endorphin:
1. Synthesis and purification are possible and practical.
2. The β-endorphin ester can be administered parenterally without adverse effects.
3. Esterase in the plasma and CNS can hydrolyze the prodrug.
4. The rate of esterase degradation in the plasma is slow, allowing the prodrug to cross the BBB.
5. The prodrug is hydrolyzed in the CNS to β-endorphin and a metabolite of known low toxicity in the human CNS.
6. β-endorphin administration in the CNS is a potent analgesic without respiratory depression, tolerance, physical dependence or addiction and devoid of any major side effects.

### Prior Art

Prior art in humans and animals teaches the following:
1. In humans, local anesthetic esters cross the BBB.
2. In humans, heroin, a diester of morphine, crosses the BBB.
3. In humans, sobetirome, a thyromimetic ester crosses the BBB.
4. In humans, fosphenytoin, a phosphate ester, crosses the BBB.
5. In humans, candesartan cilexetil, an antihypertensive ester, crosses the BBB.
6. In humans, triethyl citrate, a calcium chelating agent, probably crosses the BBB.[17]
7. In animals, GABA esters of glucose, dexamethasone, linolenoyl alcohol, and cholesterol cross the BBB.[18, 19]
8. In animals, opioid peptides esters (2-3 amino acids) likely cross the BBB.[20]
9. In animals, glucosyl esters of ketoprofen and indomethacin cross the BBB.
10. In animals, dehydroascorbic acid crosses via GLUT1 transporter the BBB.[21]
11. In animals, opioid glycopeptides cross the BBB.

### Non-Opioid Nociceptive Pathways

Pain involves redundant opioid and non-opioid pathways. Chronic pain sufferers often require a combination of opioids and other medications such as tricyclic antidepressants, anticonvulsants, and anti-inflammatory drugs. This invention offers an improvement, not a complete solution, for pain treatment.

### Pharmaceutical Incentives

The development of a prodrug of β-endorphin may not be proprietary, which could result in limited financial incentives for pharmaceutical companies to conduct this research. Publications and patent applications have already outlined opioid peptide esters, suggesting that the esterification of β-endorphin might not be considered novel or non-obvious.[20, 24] As a result, direct government funding for this translational scientific research may be required.

### BRIEF DESCRIPTION OF THE DRAWINGS

FIG. 1 is the genetic profile of the amino acid sequence of the μ-opioid receptor in humans, mice and rats. OPMR_MOUSE is SEQ ID NO: 1, OPMR_RAT is SEQ ID NO: 2, and OPMR_HUMAN is SEQ ID NO: 3. No new matter has been added to the specification and drawings.

FIG. 2 is the genetic profile of the amino acid sequence of β-endorphin in humans, mice, rats, and camels. COLI_HUMAN is SEQ ID NO: 4, COLI_RAT is SEQ ID NO: 5, COLI_MOUSE is SEQ ID NO: 6, and COLI_CAMDR is SEQ ID NO: 7. No new matter has been added to the specification and drawings.

FIG. 3 is docking of β-endorphin to the μ-opioid receptor. Label 1 is the alpha helix of β-endorphin. Label 2 is the μ-opioid receptor.

FIG. 4 is docking of morphine to the μ-opioid receptor.

### BRIEF SUMMARY OF THE INVENTION

The safe and effective treatment of chronic intractable pain remains an unmet need, with years of research failing to develop a potent μ-agonist possessing a favorable side effect profile. In humans exogenous intrathecal and epidural administration of β-endorphin has demonstrated profound analgesia without respiratory depression and with minimal adverse effects of tolerance, physical dependence, and addiction. It is hypothesized, based on observations of excess endocrinopathies, that sustained GPCR stimulation from endogenous ligands usually does not produce tolerance via desensitization from β-arrestin translocation, intracellular phosphorylation, or internalization. This suggests that sustained endogenous ligand GPCR binding is receptor-biased, which is likely true for β-endorphin. Currently, computational chemistry cannot design a μ-agonist superior to natural compounds. It is proposed that selective esterification of β-endorphin with lipophilic molecules and/or active transport molecules known to be familiar and generally nontoxic to the CNS such as ethanol, cholesterol, glucose, DHA, linolenic acid, or ascorbic acid will yield a prodrug capable of crossing the human BBB and undergoing esterase hydrolysis, resulting in a safe and potent analgesic. The sites of esterification include the carboxylic acids of the C-terminal glutamic acid (31).

### DETAILED DESCRIPTION OF THE INVENTION

It is proposed that β-endorphin serves as a GPCR-biased μ-agonist capable of producing profound analgesia without causing major side effects of respiratory depression, tolerance, physical dependence, and addiction. Furthermore, it is hypothesized that esterification of β-endorphin will enhance its lipophilicity as a prodrug, enabling it to cross the human BBB and subsequently be hydrolyzed by CNS esterases and metabolized into β-endorphin and ethanol, cholesterol, DHA, linolenic acid and/or esterification of β-endorphin with an actively transported molecule such as glucose, DHA, dehydroascorbic acid will enable it to cross the BBB.

Prior art teaches that peptides can cross the BBB without meeting Lipinski criteria. Prior art also teaches that prodrugs with ester functional groups can increase a drug's lipophilicity allowing drugs to cross the BBB and be hydrolyzed by CNS esterases to active drug and a metabolite that is known to have very low if any toxicity within the CNS. In human case studies of patients with intractable cancer pain administration of intrathecal β-endorphin produces profound analgesia without respiratory depression or tolerance.

### Computational Chemistry

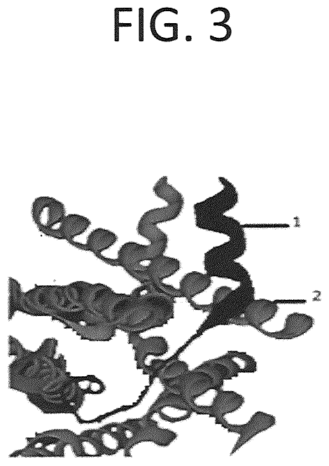

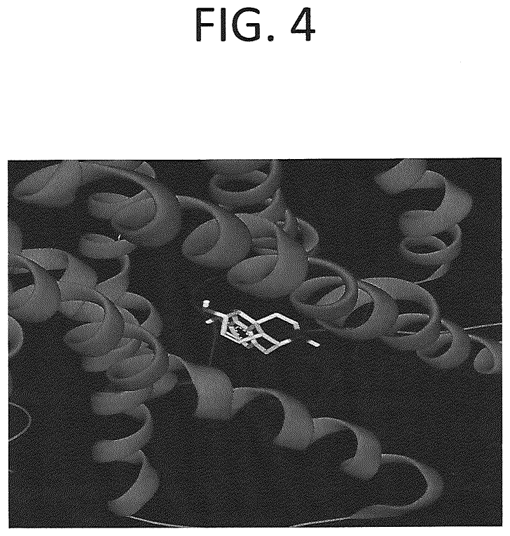

The μ receptor has been subdivided into μ1, μ2, and μ3 receptors and the μ3 receptor is not responsive to opioid peptides but responsive to opioid alkaloids. Therefore, there is discrimination between an alkaloid ligand and opioid peptide binding to a GPCR, and this is supported by molecular docking. (FIGS. 3-4) The μ2 receptor has TRMU-5 agonists properties producing a different analgesic response.

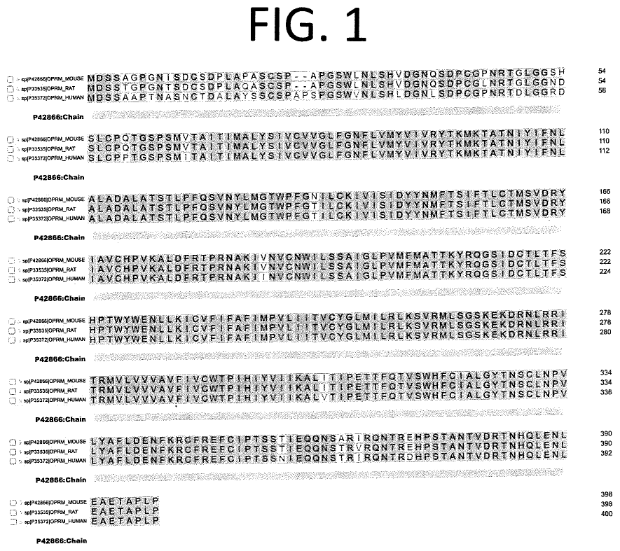

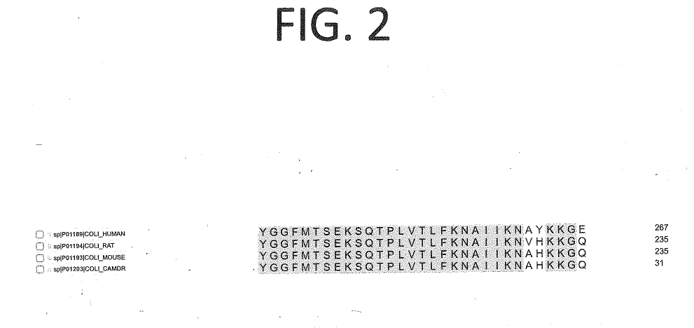

The amino acid sequence for the μ1 receptor is close but not identical in humans, mice and rats. (FIG. 1) The amino acid sequence for β-endorphin is not identical in humans, camels, mice and rats. (FIG. 2) Therefore, earlier work that established tolerance and respiratory depression with administration of β-endorphin in animal models may not be directly translated to humans.

Esterification of carboxylic acids to esters is well known to increase lipophilicity of a drug. The Lipinski criteria for glutamic acid, ethyl glutamate, diethyl glutamate and morphine, 3-acetylmorphine, diacetylmorphine (heroin) are shown in Tables 3 and 4.

TABLE 3
Lipinski Comparison of Glutamic Acid,
Ethyl Glutamate, and Diethyl Glutamate

| | XlogP3 | MW | HBD | HBA | Rot. Bonds |
|---|---|---|---|---|---|
| Glutamic Acid | −3.7 | 147 | 3 | 5 | 4 |
| Ethyl Glutamate | −3 | 175 | 2 | 5 | 6 |
| Diethyl Glutamate | 0.2 | 203 | 1 | 5 | 8 |

TABLE 4
Lipinski Comparison of Morphine, 3-
Acetylmorphine, and Diacetylmorphine

| | XlogP3 | MW | HBD | HBA | Rot. Bonds |
|---|---|---|---|---|---|
| Morphine | 0.8 | 285 | 2 | 4 | 0 |
| 3-Acetylmorphine | 0.9 | 327 | 1 | 5 | 2 |
| Diacetylmorphine | 1.5 | 369 | 0 | 6 | 4 |

The addition of a moiety that is relatively nontoxic to the CNS which forms a conjugate or prodrug with β-endorphin can be rated based upon lipophilicity (X log P3) and potential to be actively transported across the human BBB as shown in Table 5.

TABLE 5
Lipinski Comparison of Conjugate Moieties Including
Active Transport

| | | Active | | | Rot. | |
|---|---|---|---|---|---|---|
| | XlogP3 | Transport | HBD | HBA | Bonds | MW |
| DHA | 6.2 | + protein | 1 | 2 | 2 | 174 |
| Linolenic acid | 5.9 | + lipid | 1 | 1 | 13 | 278 |
| Cholesterol | 8.7 | + lipid | 1 | 1 | 5 | 386 |
| Ethanol | −0.1 | — | 1 | 1 | 0 | 46 |
| Glucose | −2.6 | + GLUT | 5 | 6 | 1 | 180 |
| Dehydroascorbic acid | −1 | + GLUT | 2 | 6 | 2 | 174 |

### Synthesis of β-Endorphin Esters

There are many methods to synthesize β-endorphin esters also referred to as a conjugate. [25] This invention describes two such methods:

Initial esterification of L-glutamic acid which may require protecting groups. Subsequent peptide conjugation of the L-glutamic ester with β-endorphin (1-30) the fragment that has been produced by cleavage of the C-terminal Glu with Endoproteinase Glu-C.

### β-Endorphin Esters Via Peptide Conjugation

Step #1. L-glutamic acid is refluxed in a suitable solvent which may be acetone with a suitable catalyst which may be HCl or chlorotrimethylsilane (TMSCI) to form the L-glutamate esters. TMSCI may promote a single ester and preferentially the alpha carbon ester.[26] Protecting groups may be required for the synthesis of the esters. Amino groups can be protected by forming a t-butoxy carbonyl derivative and following esterification protecting groups can be removed by standard techniques for example treatment with an acid. Ethyl alcohol, cholesterol, linolenoyl alcohol, and glucose can be directly esterified. DHA is reduced to alcohol and then esterified. Ascorbic acid is oxidized to dehydroascorbic acid and then esterified After reflux the solvent is evaporated and the residual extracted with diethyl ether or chloroform or a similar solvent. The glutamate esters are isolated and purified by methods that may include chromatography, electrophoresis, and/or crystallization. Step #2. β-endorphin is dissolved in a cleavage buffer which could be hydroxylamine HCl or Tris-HCl and with addition of Endoproteinase Glu-C. The β-endorphin (1-30) fragment is isolated and purified. Step #3. The β-endorphin (1-30) C-terminal is conjugated to the glutamic acid ester with a peptide bond by solid phase synthesis or similar methods. The β-endorphin ester is isolated and purified by methods which may include chromatography, electrophoresis, and/or crystallization yielding conjugates (1-12).

### Fischer Esterification of the C-Terminal Glutamate (31)

The initial esterification of β-endorphin with ethyl alcohol, cholesterol, glucose, docosahexaenoly alcohol, linolenoyl alcohol, or dehydroascorbic acid is achieved through Fischer esterification of the C-terminal carboxylic acid of glutamic acid (31) or carboxylic acid side chain of the C-terminal glutamic acid (31) resulting in the formation of conjugates 1-12. A suitable solvent may be the reagent alcohol or acetone. A suitable catalyst may be HCl. The solution is evaporated to produce a solid residue which is extracted with diethyl ether or chloroform or similar solvent and water. The organic extract is evaporated and purified by chromatography, electrophoresis, and/or crystallization yielding conjugates (1-12)

Protecting groups may be required for the synthesis of the conjugates; however, OH groups can be protected by acetonide derivatives and amino groups can be protected by forming a t-butoxy carbonyl derivative and following esterification protecting groups can be removed by standard techniques for example treatment with an acid.

It is assumed that the carboxylic acid of glutamic acid (8) is sterically hindered and will not participate in the esterification process.

It is assumed that the Fischer esterification of the conjugates will not produce a diester but will produce a mixture of esters at the C-terminal Glu (31) including esterification at the terminal carboxylic acid or at the Glu (31) side chain carboxylic acid and these conjugates can be separated and purified by chromatography, electrophoresis, and/or crystallization. The relative proportions of these esters may be favored by increasing or decreasing concentrations of reactants and dependent upon temperature.

It is assumed that Fischer esterification will successfully produce the conjugates; however, β-endorphin esters can be prepared by converting β-endorphin to the anhydride and subsequent esterification of the anhydride. Furthermore β-endorphin esters may be prepared by other methods know in the art as taught in U.S. Pat. No. 5,051,448.

Conjugate 1 (β-endorphin C-terminal α-ethyl ester) Tyr-Gly-Gly-Phe-Met-Thr-
Ser-Glu-Lys-Ser-Gln-Thr-Pro-Leu-Val-Thr-Leu-Phe-Lys-Asn-Ala-Ile-Ile-Lys-Asn-Ala-Tyr-Lys-
Lys-Gly-Glu-α-Ethyl
Conjugate 2 (β-endorphin C-terminal γ-ethyl ester) Tyr-Gly-Gly-Phe-Met-Thr-
Ser-Glu-Lys-Ser-Gln-Thr-Pro-Leu-Val-Thr-Leu-Phe-Lys-Asn-Ala-Ile-Ile-Lys-Asn-Ala-Tyr-Lys-
Lys-Gly-Glu-γ-Ethyl
Conjugate 3 (β-endorphin C-terminal α-cholesteryl ester) Tyr-Gly-Gly-Phe-Met-
Thr-Ser-Glu-Lys-Ser-Gln-Thr-Pro-Leu-Val-Thr-Leu-Phe-Lys-Asn-Ala-Ile-Ile-Lys-Asn-Ala-Tyr-
Lys-Lys-Gly-Glu- α-Cholesteryl
Conjugate 4 (β-endorphin C-terminal γ-cholesteryl ester) Tyr-Gly-Gly-Phe-Met-
Thr-Ser-Glu-Lys-Ser-Gln-Thr-Pro-Leu-Val-Thr-Leu-Phe-Lys-Asn-Ala-Ile-Ile-Lys-Asn-Ala-Tyr-
Lys-Lys-Gly-Glu- γ-Cholesteryl
Conjugate 5 (β-endorphin C-terminal α-3-glucosyl ester) Tyr-Gly-Gly-Phe-Met-
Thr-Ser-Glu-Lys-Ser-Gln-Thr-Pro-Leu-Val-Thr-Leu-Phe-Lys-Asn-Ala-Ile-Ile-Lys-Asn-Ala-Tyr-
Lys-Lys-Gly-Glu- α-3-Glucosyl
Conjugate 6 (β-endorphin C-terminal γ-3-glucosyl ester) Tyr-Gly-Gly-Phe-Met-
Thr-Ser-Glu-Lys-Ser-Gln-Thr-Pro-Leu-Val-Thr-Leu-Phe-Lys-Asn-Ala-Ile-Ile-Lys-Asn-Ala-Tyr-
Lys-Lys-Gly-Glu-γ-3-Glucosyl
Conjugate 7 (β-endorphin C-terminal α-docosahexaenoyl ester) Tyr-Gly-Gly-
Phe-Met-Thr-Ser-Glu-Lys-Ser-Gln-Thr-Pro-Leu-Val-Thr-Leu-Phe-Lys-Asn-Ala-Ile-Ile-Lys-
Asn-Ala-Tyr-Lys-Lys-Gly-Glu-α-Docosahexaenoyl
Conjugatge 8 (β-endorphin C-terminal γ-docosahexaenoyl ester) Tyr-Gly-Gly-
Phe-Met-Thr-Ser-Glu-Lys-Ser-Gln-Thr-Pro-Leu-Val-Thr-Leu-Phe-Lys-Asn-Ala-Ile-Ile-Lys-
Asn-Ala-Tyr-Lys-Lys-Gly-Glu-γ-Docosahexaenoyl
Conjugate 9 (β-endorphin C-terminal α-linolenoyl ester) Tyr-Gly-Gly-Phe-Met-
Thr-Ser-Glu-Lys-Ser-Gln-Thr-Pro-Leu-Val-Thr-Leu-Phe-Lys-Asn-Ala-Ile-Ile-Lys-Asn-Ala-Tyr-
Lys-Lys-Gly-Glu- α-Linolenoyl
Conjugate 10 (β-endorphin C-terminal γ-linolenoyl ester) Tyr-Gly-Gly-Phe-Met-
Thr-Ser-Glu-Lys-Ser-Gln-Thr-Pro-Leu-Val-Thr-Leu-Phe-Lys-Asn-Ala-Ile-Ile-Lys-Asn-Ala-Tyr-
Lys-Lys-Gly-Glu- γ-Linolenoyl
Conjugate 11 (β-endorphin C-terminal α-dehydroascorbate) Tyr-Gly-Gly-Phe-
Met-Thr-Ser-Glu-Lys-Ser-Gln-Thr-Pro-Leu-Val-Thr-Leu-Phe-Lys-Asn-Ala-Ile-Ile-Lys-Asn-
Ala-Tyr-Lys-Lys-Gly-Glu-α-Dehydroascorbate
Conjugate 12 (β-endorphin C-terminal γ-dehydroascorbate) Tyr-Gly-Gly-Phe-
Met-Thr-Ser-Glu-Lys-Ser-Gln-Thr-Pro-Leu-Val-Thr-Leu-Phe-Lys-Asn-Ala-Ile-Ile-Lys-Asn-
Ala-Tyr-Lys-Lys-Gly-Glu-γ-Dehydroascorbate

### REFERENCES

2. Lambert, D. and G. Calo, Approval of oliceridine (TRV130) for intravenous use in moderate to severe pain in adults. Br J Anaesth, 2020. 125(6): p. e473-e474.
3. Smyth, D.G., 60 YEARS OF POMC: Lipotropin and beta-endorphin: a perspective. J Mol Endocrinol, 2016. 56(4): p. T13-25.
4. Oyama, T., et al., Profound analgesic effects of beta-endorphin in man. Lancet, 1980. 1(8160): p. 122-4.
5. Oyama, T., S. Fukushi, and T. Jin, Epidural beta-endorphin in treatment of pain. Can Anaesth Soc J, 1982. 29(1): p. 24-6.
6. Jin, T., T. Murakawa, and T. Oyama, [Analgesic effect of continuous intrathecal beta-endorphin in cancer patients]. Masui, 1986. 35(1): p. 148-51.
7. Xu, J., et al., Alternatively spliced mu opioid receptor C termini impact the diverse actions of morphine. J Clin Invest, 2017. 127(4): p. 1561-1573.
8. Tive, L.A., et al., Analgesic potency of TRIMU-5: a mixed mu 2 opioid receptor agonist/mu 1 opioid receptor antagonist. Eur J Pharmacol, 1992. 216(2): p. 249-55.
9. Cadet, P., K.J. Mantione, and G.B. Stefano, Molecular identification and functional expression of mu 3, a novel alternatively spliced variant of the human mu opiate receptor gene. J Immunol, 2003. 170(10): p. 5118-23.
10. Banks, W.A., Viktor Mutt lecture: Peptides can cross the blood-brain barrier. Peptides, 2023. 169: p. 171079.
11. Moss, I.R. and E. Friedman, beta-Endorphin: effects on respiratory regulation. Life Sci, 1978. 23(12): p. 1271-6.
12. Moss, I.R. and E.M. Scarpelli, beta-Endorphin central depression of respiration and circulation. J Appl Physiol Respir Environ Exerc Physiol, 1981. 50(5): p. 1011-6.
13. Sitsen, J.M., J.M. Van Ree, and W. De Jong, Cardiovascular and respiratory effects of beta-endorphin in anesthetized and conscious rats. J Cardiovasc Pharmacol, 1982. 4(6): p. 883-8.
14. Tseng, L.F., H.H. Loh, and C.H. Li, Human beta-endorphin: development of tolerance and behavioral activity in rats. Biochem Biophys Res Commun, 1977. 74(2): p. 390-6.
15. van Ree, J.M., et al., Induction of tolerance to the analgesic action of lipotropin in C-fragment. Nature, 1976. 264(5588): p. 792-4.
16. Hosobuchi, Y., et al., beta-Endorphin: development of tolerance and its reversal by 5-hydroxytryptophan in cats. Proc Natl Acad Sci U S A, 1977. 74(9): p. 4017-9.
17. Goldberg, J.S., MEDICINAL PROPERTIES OF TRIETHYL CITRATE U.S. Pat. No. 11,786,495 B2. 2023.
18. Shashoua, V.E., GABA ESTERS AND GABA ANALOG ESTERS U.S. Pat. No. 5,051,448. 1991.
19. Goldberg, J.S., Selected Gamma Aminobutyric Acid (GABA) Esters may Provide Analgesia for Some Central Pain Conditions. Perspect Medicin Chem, 2010. 4: p. 23-31.
20. Goldberg, J.S., OPIOID PEPTIDE ESTERS AND METHODS OF USE US 2013/0072438 A1.
21. Agus, D.B., et al., Vitamin C crosses the blood-brain barrier in the oxidized form through the glucose transporters. J Clin Invest, 1997. 100 (11): p. 2842-8.
22. Polt, R., et al., Glycopeptide enkephalin analogues produce analgesia in mice: evidence for penetration of the blood-brain barrier. Proc Natl Acad Sci U S A, 1994. 91(15): p. 7114-8.
23. Goldberg, J.S., Chronic opioid therapy and opioid tolerance: a new hypothesis. Pain Res Treat, 2013. 2013: p. 407504.
24. Goldberg, J.S., Low Molecular Weight Opioid Peptide Esters Could be Developed as a New Class of Analgesics. Perspect Medicin Chem, 2011. 5: p. 19-26.
25. Goldberg, J.S., NOVEL SYNTHESIS OF POTENTIAL ESTER PRODRUGS US 2017/0165371 A1.
26. Takaishi, T., et al., Product Selectivity of Esterification of L-Aspartic Acid and L-Glutamic Acid Using Chlorotrimethylsilane. Nat Prod Commun, 2017. 12(2): p. 247-249.

Having described my invention, I claim:

1. A conjugate comprising:
β-endorphin; and
a moiety conjugated to a C-terminal carboxylic acid of a glutamic acid of a β-endorphin by an ester bond, wherein the moiety comprises ethyl alcohol, cholesterol, glucose, docosahexaenoyl alcohol, linolenoyl alcohol, or dehydroascorbic acid.

2. The conjugate of claim 1, wherein the conjugate comprises:
a. Tyr-Gly-Gly-Phe-Met-Thr-Ser-Glu-Lys-Ser-Gln-Thr-Pro-Leu-Val-Thr-Leu-Phe-
Lys-Asn-Ala-Ile-Ile-Lys-Asn-Ala-Tyr-Lys-Lys-Gly-Glu-α-Ethyl (β-endorphin C-
terminal α-ethyl ester)
b. Tyr-Gly-Gly-Phe-Met-Thr-Ser-Glu-Lys-Ser-Gln-Thr-Pro-Leu-Val-Thr-Leu-Phe-
Lys-Asn-Ala-Ile-Ile-Lys-Asn-Ala-Tyr-Lys-Lys-Gly-Glu-α-Cholesteryl (β-endorphin
C-terminal α-cholesteryl ester)
c. c. Tyr-Gly-Gly-Phe-Met-Thr-Ser-Glu-Lys-Ser-Gln-Thr-Pro-Leu-Val-Thr-Leu-Phe-
Lys-Asn-Ala-Ile-Ile-Lys-Asn-Ala-Tyr-Lys-Lys-Gly-Glu-α-3-Glucosyl (β-endorphin
C-terminal α-3-glucosyl ester)
d. Tyr-Gly-Gly-Phe-Met-Thr-Ser-Glu-Lys-Ser-Gln-Thr-Pro-Leu-Val-Thr-Leu-Phe-
Lys-Asn-Ala-Ile-Ile-Lys-Asn-Ala-Tyr-Lys-Lys-Gly-Glu-α-Docosahexaenoyl (β-
endorphin C-terminal α-docosahexaenoyl ester)
e. Tyr-Gly-Gly-Phe-Met-Thr-Ser-Glu-Lys-Ser-Gln-Thr-Pro-Leu-Val-Thr-Leu-Phe-
Lys-Asn-Ala-Ile-Ile-Lys-Asn-Ala-Tyr-Lys-Lys-Gly-Glu-α-Linolenoyl (β-endorphin
C-terminal α-linolenoyl ester)
f. Tyr-Gly-Gly-Phe-Met-Thr-Ser-Glu-Lys-Ser-Gln-Thr-Pro-Leu-Val-Thr-Leu-Phe-
Lys-Asn-Ala-Ile-Ile-Lys-Asn-Ala-Tyr-Lys-Lys-Gly-Glu-α-Dehydroascorbate (β-
endorphin C-terminal α-dehydroascorbate).

3. The conjugate of claim 1, wherein the moiety conjugated to the C-terminal carboxylic acid is the moiety conjugated to a side chain carboxylic acid.

4. The conjugate of claim 3, wherein the conjugate comprises:
a. Tyr-Gly-Gly-Phe-Met-Thr-Ser-Glu-Lys-Ser-Gln-Thr-Pro-Leu-Val-Thr-Leu-Phe-
Lys-Asn-Ala-Ile-Ile-Lys-Asn-Ala-Tyr-Lys-Lys-Gly-Glu-γ-Ethyl (β-endorphin C-
terminal γ-ethyl ester)
b. Tyr-Gly-Gly-Phe-Met-Thr-Ser-Glu-Lys-Ser-Gln-Thr-Pro-Leu-Val-Thr-Leu-Phe-
Lys-Asn-Ala-Ile-Ile-Lys-Asn-Ala-Tyr-Lys-Lys-Gly-Glu-γ-Cholesteryl (β-endorphin
C-terminal γ-cholesteryl ester)
c. Tyr-Gly-Gly-Phe-Met-Thr-Ser-Glu-Lys-Ser-Gln-Thr-Pro-Leu-Val-Thr-Leu-Phe-
Lys-Asn-Ala-Ile-Ile-Lys-Asn-Ala-Tyr-Lys-Lys-Gly-Glu-γ-3-Glucosyl (β-endorphin
C-terminal γ-3-glucosyl ester)
d. Tyr-Gly-Gly-Phe-Met-Thr-Ser-Glu-Lys-Ser-Gln-Thr-Pro-Leu-Val-Thr-Leu-Phe-
Lys-Asn-Ala-Ile-Ile-Lys-Asn-Ala-Tyr-Lys-Lys-Gly-Glu-γ-Docosahexaenoyl (β-
endorphin C-terminal γ-docosahexaenoyl ester)
e. Tyr-Gly-Gly-Phe-Met-Thr-Ser-Glu-Lys-Ser-Gln-Thr-Pro-Leu-Val-Thr-Leu-Phe-
Lys-Asn-Ala-Ile-Ile-Lys-Asn-Ala-Tyr-Lys-Lys-Gly-Glu-γ-Linolenoyl (β-endorphin
C-terminal γ-linolenoyl ester)
f. Tyr-Gly-Gly-Phe-Met-Thr-Ser-Glu-Lys-Ser-Gln-Thr-Pro-Leu-Val-Thr-Leu-Phe-
Lys-Asn-Ala-Ile-Ile-Lys-Asn-Ala-Tyr-Lys-Lys-Gly-Glu-γ-Dehydroascorbate (β-
endorphin C-terminal γ-dehydroascorbate).

5. A method of altering nociception, comprising administering to a subject an effective amount of a conjugate of claim 2 thereby altering nociception in the subject.

6. A method of altering nociception, comprising administering to a subject an effective amount of a conjugate of claim 4 thereby altering nociception in the subject.


SEQUENCE LISTING (7 sequences)

SEQ ID NO: 1 - protein, 398 aa, Mus musculus
MDSSAGPGNISDCSDPLAPASCSPAPGSWLNLSHVDGNQSDPCGPNRTGLGGSHSLCPQTGSPSMVTAITIMALYSIVCVVGLFGNFLVMYVIVRYTKMKTATNIYIFNLALADALATSTLPFQSVNYLMGTWPFGNILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSARIRQNTREHPSTANTVDRTNHQLENLEAETAPLP

SEQ ID NO: 2 - protein, 398 aa, Rattus norvegicus
MDSSTGPGNTSDCSDPLAQASCSPAPGSWLNLSHVDGNQSDPCGLNRTGLGGNDSLCPQTGSPSMVTAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSTRVRQNTREHPSTANTVDRTNHQLENLEAETAPLP

SEQ ID NO: 3 - protein, 400 aa, Homo sapiens
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP

SEQ ID NO: 4 - protein, 31 aa, Homo sapiens
YGGFMTSEKSQTPLVTLFKNAIIKNAYKKGE

SEQ ID NO: 5 - protein, 31 aa, Rattus norvegicus
YGGFMTSEKSQTPLVTLFKNAIIKNVHKKGQ

SEQ ID NO: 6 - protein, 31 aa, Mus musculus
YGGFMTSEKSQTPLVTLFKNAIIKNAHKKGQ

SEQ ID NO: 7 - protein, 31 aa, Camelus dromedarius; identical to SEQ ID NO: 6